SEQUENCE LISTING (11 sequences)

SEQ ID NO: 1 - DNA, 19 bp, Artificial Sequence
  synthetic GAPDH500Fp-1 primer
agccacaucgcucagacac

SEQ ID NO: 2 - DNA, 21 bp, Artificial Sequence
  synthetic GAPDH500R primer
gaggcattgctgatgatcttg

SEQ ID NO: 3 - DNA, 19 bp, Artificial Sequence
  synthetic GAPDH500Fp-2 primer
  misc_feature (8)..(8): n refers to hypoxanthine nucleotide (I).
  misc_feature (12)..(12): n refers to hypoxanthine nucleotide (I).
agccacancgcncagacac

SEQ ID NO: 4 - DNA, 21 bp, Artificial Sequence
  synthetic GAPDH500R primer
gaggcattgctgatgatcttg

SEQ ID NO: 5 - DNA, 472 bp, Artificial Sequence
  synthetic template DNA-1
agccacaucgcucagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctc

SEQ ID NO: 6 - DNA, 472 bp, Artificial Sequence
  synthetic template DNA-2
  misc_feature (8)..(8): n refers to hypoxanthine nucleotide (I).
  misc_feature (12)..(12): n refers to hypoxanthine nucleotide (I).
agccacancgcncagacaccatggggaaggtgaaggtcggagtcaacggatttggtcgtattgggcgcctggtcaccagggctgcttttaactctggtaaagtggatattgttgccatcaatgaccccttcattgacctcaactacatggtttacatgttccaatatgattccacccatggcaaattccatggcaccgtcaaggctgagaacgggaagcttgtcatcaatggaaatcccatcaccatcttccaggagcgagatccctccaaaatcaagtggggcgatgctggcgctgagtacgtcgtggagtccactggcgtcttcaccaccatggagaaggctggggctcatttgcaggggggagccaaaagggtcatcatctctgccccctctgctgatgcccccatgttcgtcatgggtgtgaaccatgagaagtatgacaacagcctcaagatcatcagcaatgcctc

SEQ ID NO: 7 - DNA, 24 bp, Artificial Sequence
  synthetic GAPDH500splint primer
agcgatgtggctgaggcattgctg

SEQ ID NO: 8 - DNA, 58 bp, Artificial Sequence
  synthetic capture sequence
  misc_feature (27)..(36): n is a, c, g or t
  misc_feature (58)..(58): V is a, c or g
aagcuucgtagccatgtcgttctgcgnnnnnnnnnntttttttttttttttttttttv

SEQ ID NO: 9 - DNA, 27 bp, Artificial Sequence
  synthetic TSO primer
  misc_feature (25)..(27): G at positions 25-27 are guanine ribonucleotides.
aagcuucgtagccatgtcgttctgggg

SEQ ID NO: 10 - DNA, 24 bp, Artificial Sequence
  synthetic ISO primer
  misc_feature (12)..(12): n refers to hypoxanthine nucleotide (I).
  misc_feature (18)..(18): n refers to hypoxanthine nucleotide (I).
aagcuucgtagncatgtngttctg

SEQ ID NO: 11 - DNA, 16 bp, Artificial Sequence
  oligo dT
tttttttttttttttt

(19) United States
(12) Patent Application Publication
(10) Pub. No.: US 2024/0279724 A1
(43) Pub. Date: Aug. 22, 2024

(54) METHOD FOR OBTAINING DOUBLE-STRANDED SEQUENCE BY SINGLE-STRANDED ROLLING CIRCLE AMPLIFICATION

(71) Applicant: BGI SHENZHEN, Shenzhen (CN)

(72) Inventors: Ji Wang, Shenzhen (CN); Tao Tan, Shenzhen (CN); Lin Zhou, Shenzhen (CN); Ou Wang, Shenzhen (CN); Wenwei Zhang, Shenzhen (CN); Ao Chen, Shenzhen (CN)

(21) Appl. No.: 18/570,440

(22) PCT Filed: Jun. 16, 2021
(86) PCT No.: PCT/CN2021/100436

§ 371 Date: Dec. 14, 2023

(51) Int. Cl. C12Q 1/6844; C12Q 1/34; C12Q 1/527; C40B 50/06

(52) U.S. Cl. CPC C12Q 1/6844; C12Q 1/34; C12Q 1/527; C40B 50/06

(57) ABSTRACT

Provided is a method for obtaining a double-stranded sequence by single-stranded rolling circle amplification, comprising: 1) performing rolling circle amplification reaction on single-stranded circular DNA by means of a first primer to obtain an amplified sequence, the first primer being complementary to a partial region of the single-stranded circular DNA, and the single-stranded circular DNA having a break mechanism that can cause the single-stranded circular DNA to ring-open; 2) ring-opening the single-stranded circular DNA by means of the break mechanism to obtain single-stranded linear DNA; and 3) using the single-stranded linear DNA as a second primer and using the amplified sequence obtained in step 1) as a template to perform amplification reaction to obtain an amplified double-stranded sequence.

### FIELD

The present disclosure relates to the technical field of biotechnology, and in specific to a method for obtaining a double-stranded sequence by single-stranded rolling circle amplification.

### BACKGROUND

Rolling circle amplification (RCA) or rolling circle replication (RCR) generally refers to subjecting a single-stranded circular DNA molecule as a template to polymerase chain reaction (PCR) with use of a DNA polymerase possessing strand displacement activity (e.g., Phi29 polymerase, Bst polymerase, Bsu polymerase, Klenow Fragment, Vent polymerase, Pol III polymerase, etc.). Usually, under appropriate reaction conditions, in response to adding a DNA or RNA fragment complementary to the sequence of a particular single-stranded circular DNA molecule, the DNA polymerases possessing strand displacement activity will take the added DNA or RNA fragment as a primer, and take the single-stranded circular DNA molecule as a template, to amplify the DNA template. As said DNA polymerase lacks 5′->3′ exonuclease activity, when a cycle of amplification ends, the polymerase unzips the DNA double strands in its forward direction to continue the amplification, resulting in a single-stranded DNA molecule containing a plurality of identical copies. Similarly, when a double-stranded circular DNA molecule (e.g., a plasmid, a viral DNA molecule, etc.) has a disconnection or a base deletion in one of the strands, the RCA as described above may also be performed.

RCA, involving the DNA polymerases with strand displacement activity, are also referred to isothermal rolling circle amplification because of its advantages such as fast reaction speed, high fidelity and constant reaction temperature, and has been widely applied. Similarly, non-circular DNA molecules may also be performed with the isothermal amplification based on the discussed mechanism. When the RCA lasts long enough, the generated single stranded DNA will fold and intertwine therebetween, forming a complexly spatial secondary structure, which coordinates with metal ions to form a structurally dense DNA nanoball (DNB), also known as DNA nanoparticle (DNP), DNA nanoflower (DNF), etc. DNB has many properties differ from conventional DNA molecules, such as the resistance to DNase digestion, the ability of forming colloid with metal ions, high cellular affinity and the like.

Although having the above mentioned advantages, DNB, as the products of RCA, is mainly composed of the single-stranded DNA molecule, which still has significant limitations in terms of stability and diversity of enzyme reactions compared to double-stranded DNA molecules. Therefore, it will greatly expand the application of RCA by converting single-stranded products of RCA into double-stranded products, and can also achieve complementary effects through joint application with other technologies, thereby promoting technological upgrading.

Several existing solutions for the conversion of single-stranded products of RCA into double-stranded products are briefly described below:
i) Side reaction of RCA. The widely used amplification polymerases in RCA are Phi29 and its mutants. Phi29 has strong strand displacement activity and certain template switch activity, i.e., during, especially at the initial stage of a reaction, the Phi29 in the position of the replication fork has a certain probability to displace to the nearby single-stranded DNA and taking this DNA as a template to replicate in a opposite direction of the initial direction of the RCA, and to finally form a double-stranded DNA product. However, although such a product of RCA is a double-stranded DNA product, generally it has a low yield as being by-products of RCA. Moreover, because this side reaction tends to occur at the initial stage of the reaction, the generated double-stranded product of RCA is shorter in length and has less amplification, and thus cannot be effectively utilized.
ii) RCA with double primers. Double-primed RCA refers to RCA whose reaction system includes a reverse primer complementary to the single-stranded circular DNA and a forward primer consistent with the sequence of the template at the same time. The reverse primer in the reaction system, which is complementary to the sequence of the template, will bind to the single-stranded circular DNA at first to initiate RCA. Then the forward primer consistent with the sequence of the template will bind to the generated RCA products, which will be used as a further template to continue to extend under the action of Phi29, then forming double-stranded RCA products. The process above is called secondary strand synthesis of DNB. Similarly, with the extending of Phi29, the 5′ end of the newly generated secondary strand product will be displaced with the strand displacement activity of Phi29, and the reverse primer in the reaction system binds to the secondary strand product after displacement, to continue extending under the action of Phi29, and so on, thus forming a complexly double-stranded DNA mesh structure. The double-primed RCA system is one of the most commonly used isothermal amplification systems. It takes advantages of producing a large number of double-stranded DNA copies in a short time and yielding a relatively stable amplification product. However, this method is also of obviously disadvantages in that, because it is difficult to precisely control the amount of each primer added, the RCA will be continuously repeated in different branched strands, which, on the one hand, makes the structure of the whole double-stranded DNB extremely complicated; and on the other hand, leads to incomplete synthesis for a lot of secondary strands due to the limitation of the spatial structure, resulting in a DNB structure with single and double strands coexisting. That makes qualification and quantification both more difficult.
iii) RCA with random primer (R2C2, Rolling Circle Amplification to Concatemeric Consensus Method). A method of random-primed RCA is similar to that of the double-primed RCA with differences in that: the random primer can bind to any position of the single-stranded RCA product; the double-stranded RCA product generated by the random primer RCA is more complicated; and compared to the double-primed RCA, the secondary strand synthesis is more completely. An exonuclease is required in the reaction to digest the generated unnecessary single-stranded DNA thereby ensuring generation of as many double strands DNA as possible. Although more complete double-stranded RCA products may be obtained through such a method, there are too many random primer binding sites thus forming a lot of branched structures, which still somewhat different from the conventional linear DNA molecules in the structure and the property.
iv) RCR with Loop-mediated isothermal amplification (LAMP) method. Adapters, sequences of which are complementary to each other, are added at both ends of a single-stranded DNA molecule respectively, to form a stem-loop structure. After one step of isothermal amplification, the single-stranded structure is converted into a double-stranded structure, which is then unzipped and extended with the addition of another primer complementary to the stem-loop structure. The adaptor at the 3′ end of the opened single strand will be folded intramolecularly, forming a stem-loop structure again, and with one more step of isothermal amplification, a double-stranded DNA molecule with two copies can be obtained. By repeating this process, a long double-stranded DNA molecule with repetitive sequences may be obtained. Currently, the LAMP method is the main method for obtaining a large number of long molecules with multiple copies by isothermal amplification. The advantages of this method are in its fast speed, complete double-stranded conversion and few by-products. However, as relatively more adapters and label sequences are needed in the reaction process, there is more redundant information in the generated molecules finally.
v) Hairpin RPA Amplification. The hairpin RPA amplification, proposed by Shanghai Haoqin Biotechnology Co., Ltd., refers to a method of self-assembling short molecules into long multimeric molecules, which is similar to LAMP but easier to realize. In this method, adapters with palindromic sequences ( . . . TATATATA . . . ) are connected into both ends of a target fragment. Because of the low annealing temperature of TA sequences, the adapters will unwind as the temperature gradually increases. Under an appropriate temperature, the palindromic sequences will undergo intramolecular pairing (i.e., DNA breathing), this property enables the folded and paired 3′ ends of the DNA, as new extension initiation positions, to perform intramolecular extension under the action of the amplifying enzyme, thus doubling the length of the original molecule. By repeating this process, a long double-stranded DNA molecule containing multiple copies may be obtained. Compared to LAMP, it is easier to design the adapters of this method, however the disadvantages of which lie in that the temperature control of DNA breathing needs to be adjusted, and the TA palindromic sequences produces non-specific amplification products.

In conclusion, the above existing technologies are of disadvantages as follows: a. incomplete secondary strand synthesis: the secondary strands cannot be completely synthesized in the existing RCA method, where most of the obtained double-stranded RCA products have some single-stranded Gap regions; b. complex secondary structure: in the several major methods of secondary strand synthesis above, the generated double-stranded RCA products often have complex secondary structures because the amount of primers to be added cannot be accurately controlled and they all requires a long reaction time, as a result, although the double-stranded DNA structures has been formed in essence, the performance on biochemical reaction of them is different from that of conventional double-stranded DNA molecules due to a lot of branches and single-stranded Gap regions in the complex structure; c. more by-products: as described above, as essentials like exogenous primers, exonuclease, and many adapters sequences are needed in the reaction process, numerous by-products will be accumulated in the reaction system, which may interfere with the downstream designed experiment.

### SUMMARY

The present disclosure provides a simple solution for obtaining a double-stranded RCA product to at least partially solve the problems of incomplete secondary strand DNA conversion, complex secondary structure, and numerous by-products during secondary strand synthesis of RCA in the related art.

Accordingly, in a first aspect, the present disclosure provides a method for obtaining a double-stranded sequence by single-stranded rolling circle amplification, including the following steps:
i) subjecting a single-stranded circular DNA to the rolling circle amplification with a first primer to obtain an amplified sequence, wherein the first primer is complementary to a partial region of the single-stranded circular DNA, and the single-stranded circular DNA possesses a disconnection mechanism to open the single-stranded circular DNA;
ii) opening the single-stranded circular DNA through the disconnection mechanism, to obtain a single-stranded linear DNA; and
iii) performing amplification with the single-stranded linear DNA as a second primer and the amplified sequence obtained in step i) as a template, to obtain an amplified double-stranded sequence.

In embodiments, the single-stranded circular DNA is obtained by cyclizing a DNA sample or a cDNA sample and introducing a specific base or a specific sequence into the cyclized single-stranded circular DNA by PCR or adapter connection.

In embodiments, the first primer is a DNA primer or an RNA primer.

In embodiments, the disconnection mechanism for opening the single-stranded circular DNA is to open the single-stranded circular DNA through a specific region in the single-stranded circular DNA, where the specific region is broken in response to a biochemical reaction.

In embodiments, the specific region includes the specific base and/or the specific sequence.

In embodiments, the specific base is a hypoxanthine deoxynucleotide (I), a deoxyuridine monophosphate (dU), an RNA base, an AP site, or a methylation site.

In embodiments, the specific sequence is a restriction endonuclease recognition site or a protein-specific binding site.

In embodiments, the restriction endonuclease recognition site is a region rich in AT sequences; preferably, the restriction endonuclease recognition site is a Chlamydomonas endonuclease recognition site or Neurospora crassa endonuclease recognition site.

In embodiments, the protein-specific binding site is a guide RNA recognition region of a CRISPR/Cas gene editing system, preferably a guide RNA recognition region of CRISPR/Cas9.

In embodiments, the method comprises adding a single-stranded DNA binding protein, a pyrophosphatase, and TE buffer during or after subjecting the single-stranded circular DNA to the rolling circle amplification.

In embodiments, the method comprises adding a helicase during or after subjecting the single-stranded circular DNA to the rolling circle amplification.

In embodiments, the helicase is a type A helicase unwinding in a 3′ to 5′ direction, preferably a Rep helicase, a UvrD helicase, a Heli308 helicase, a PcrA helicase, or a RecD2 helicase.

In a second aspect, the present disclosure provides a method for constructing a nucleic-acid sequencing library, including:
i) obtaining an amplified double-stranded sequence according to a method of the first aspect; and
ii) subjecting the amplified double-stranded sequence to sequencing library construction, to obtain the nucleic-acid sequencing library.

In some embodiments, the sequencing library construction is performed with long fragment read technology (LFR) to obtain the nucleic-acid sequencing library.

In some embodiments, the nucleic-acid sequencing library is an mRNA full-length transcript library.

In a third aspect, the present disclosure provides a sequencing method, including:
i) obtaining an amplified double-stranded sequence by a method according to the first aspect, or obtaining a nucleic-acid sequencing library by a method according to second aspect; and
ii) sequencing the amplified double-stranded sequence or the nucleic-acid sequencing library.

In some embodiments, the sequencing is high throughput sequencing.

In some embodiments, the sequencing is next generation sequencing or third generation sequencing.

The method in embodiments of the present disclosure has the following advantages: the method generated fewer by-products, as no exogenous primer is required in the amplification; the generated double-stranded RCA products is in a single type and without branched strands; it is easy to capture and purify the generated double-stranded RCA product; the reaction is easy to operate as it is in a single-tube; the product is more similar to the conventional DNA molecule, since the assistance of helicase reduces the complexity of the secondary structure; and the long double-stranded DNA with multi-copy generated completely may be applied to single-molecule sequencing platform, especially the ONT, because the multi-copy DNA molecule is conducive to improving the accuracy of single-molecule sequencing.

### BRIEF DESCRIPTION OF THE DRAWINGS

FIG. 1 is a schematic diagram showing (A) a single-stranded circular DNA with a specific base or specific sequence, and (B) a conventional RCA product.

FIG. 2 is a schematic diagram showing a method of obtaining a double-stranded sequence by single-stranded rolling circle amplification according to an embodiment of the present disclosure.

FIG. 3 is a schematic diagram illustrating a mechanism of a helicase in the 3′-5′ direction binding to a sequence amplified by rolling circle amplification according to an embodiment of the present disclosure.

FIG. 4 shows that a helicase affects secondary strand synthesis efficiency according to an embodiment of the present disclosure.

FIG. 5 shows agarose gel electrophoresis results of RCA products with different complex secondary structures according to an embodiment of the present disclosure.

FIG. 6 shows a relationship between coverage lengths versus coverage rate in assembly of Sample 1 according to an embodiment of the present disclosure.

FIG. 7 shows a relationship between transcript lengths versus coverage rate of assembly of Sample 1 according to an embodiment of the present disclosure.

FIG. 8 shows a distribution of the coverage lengths in assembly of Sample 1 according to an embodiment of the present disclosure.

### DETAILED DESCRIPTION

The present disclosure provides in embodiments a simple solution for obtaining a double-stranded RCA product, including: firstly, subjecting a single-stranded circular DNA to the rolling circle amplification by means of a first primer to obtain an amplified sequence; then, opening the single-stranded circular DNA through a disconnection mechanism such as a specific base or a specific sequence in the single-stranded circular DNA, to obtain a single-stranded linear DNA; lastly, performing amplification in the opposite direction of RCA by means of the single-stranded linear DNA as a second primer, to obtain an amplified double-stranded sequence.

In a specific example of the present disclosure, the method may include four steps as follows: step 1, including preparing a single-stranded circular DNA containing a specific base or sequence that enables the single-stranded circular DNA to be opened; step 2, including subjecting the single-stranded circular DNA to RCA by means of a first primer to obtain an amplified sequence, in which a helicase may be added during or after the RCA, to participate in the RCA; step 3, including disconnecting the single-stranded circular DNA at the specific base or sequence therein through a biochemical reaction, to form a single-stranded linear DNA; and step 4, including performing amplification in the opposite direction of RCA by means of the single-stranded linear DNA as a second primer, to obtain an amplified double-stranded sequence. As an example, FIG. 2 shows a method for obtaining a double-stranded sequence by single-stranded rolling circle amplification according to examples of the present disclosure, principle of which is described below, step by step.

In the step 1, the single-stranded circular DNA containing the specific base (or specific sequence) is prepared.

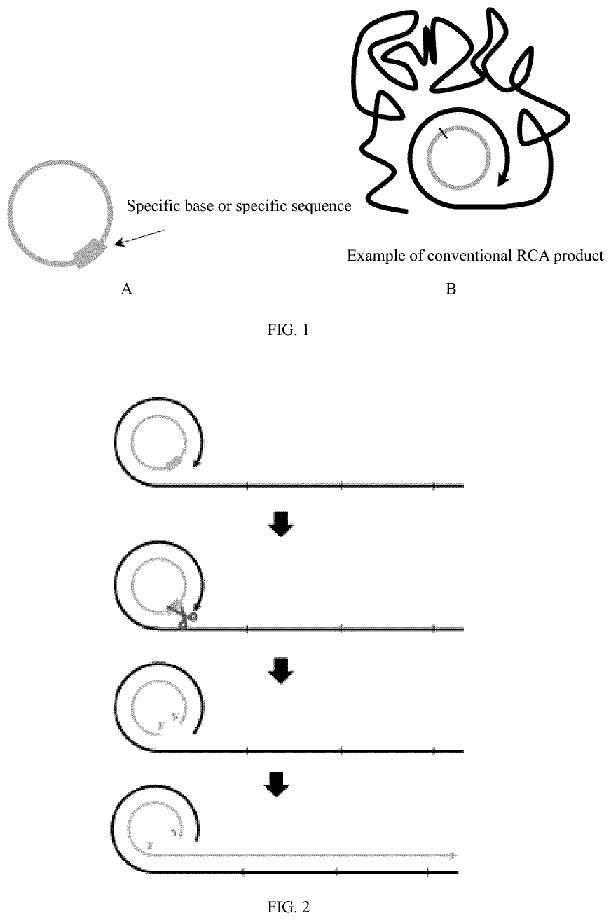

In embodiments of the present disclosure, the single-stranded circular DNA may be prepared by cycling a DNA sample directly or by cycling a cDNA sample obtained from total RNA transcription. The specific base or specific sequence is introduced into the single-stranded is circular DNA by PCR or adapter connection, thus to, if required, disconnect the single-stranded circular DNA at the specific base or specific sequence through the biochemical reaction, to open the single-stranded circular DNA, thereby obtaining the single-stranded linear DNA. In FIG. 1, portion (A) shows the single-stranded circular DNA with the specific base or specific sequence. The specific base includes, but is not limited to a base I (inosine monophosphate), a base dU (deoxyuridine monophosphate), an RNA base, an AP site, or a methylation site. The specific sequence may also be introduced, which includes, but is not limited to restriction endonuclease recognition site and so on. In examples, base I and base dU are taken as examples for opening the circular DNA, and other ring-opening methods will not be listed here.

In examples of the present disclosure, preferably, opening the single-stranded circular DNA through the specific base or specific sequence is controlled, for example, the single-stranded circular DNA is opened under conditions suitable for the biochemical reaction. For instance:
a) for a hypoxanthine nucleotide (i.e. base I), it may be digested and cleaved by endonuclease V to open the single-stranded ring;
b) base dU may be recognized and cleaved by uracil-DNA glycosylase (UDG) or apyrimidinic endonuclease 1 (APE1), to open the single-stranded ring;
c) the RNA base may be recognized and cleaved by RNaseA, RNaseH and the like, to open the single-stranded ring;
d) the AP site may be recognized and cleaved by APE1, to open the single-stranded ring;
e) the methylation site of a methylated cytosine (C) may be treated by APOBEC deaminase, ten-eleven transmethylase 2 (TET2), or sodium bisulfite to convert the methylated C to a dU, which is recognized and cleaved by UDG or APE1, to open the single-stranded ring;
f) for a region rich in AT sequences in the single-stranded ring, it may be recognized and cleaved by a Chlamydomonas endonuclease or Neurospora crassa endonuclease, to open the single-stranded ring; and
g) for a specific protein binding sequence, which may be a guide RNA recognition sequence of a CRISPR/Cas gene editing system in an example, the mutation containing CRISPR/Cas9 system induces a single-stranded break (SSB) in the DNA ring, thereby opening the single-stranded ring.

In addition to the above, any base or specific sequence that may be used to open the ring of single-stranded DNA may be taken as an alternative to this scheme to prepare for the subsequent ring-opening reaction of DNA. The present disclosure is intended to encompass any base and specific sequence that can open the single-stranded DNA.

In the step 2, RCA is performed.

The single-stranded circular DNA is subjected to RCA by means of the first primer to obtain an amplified sequence, where RCA reaction rate is very fast, and the amplified single-stranded DNA, in the role of pyrophosphate and magnesium ions, will likely to anneal, winding, folding therebetween to form complex secondary structure. In FIG. B, portion (B) shows a conventional RCA product. In order to make the RCA product's structure relatively loose, it is preferable to introduce components such as a single-stranded DNA binding protein, a pyrophosphatase and TE buffer during RCA, to obtain the RCA products with relatively “fluffy” structures. However, even with the addition of the above components, the RCA product obtained will eventually form dense DNB molecules, which is not conducive to secondary strand synthesis. On the one hand, it is difficult for primers of the secondary strands to bind to RCA products completely after RCA. On the other hand, secondary strand synthases cannot convert RCA products into complete double-stranded products due to steric hindrance and other factors.

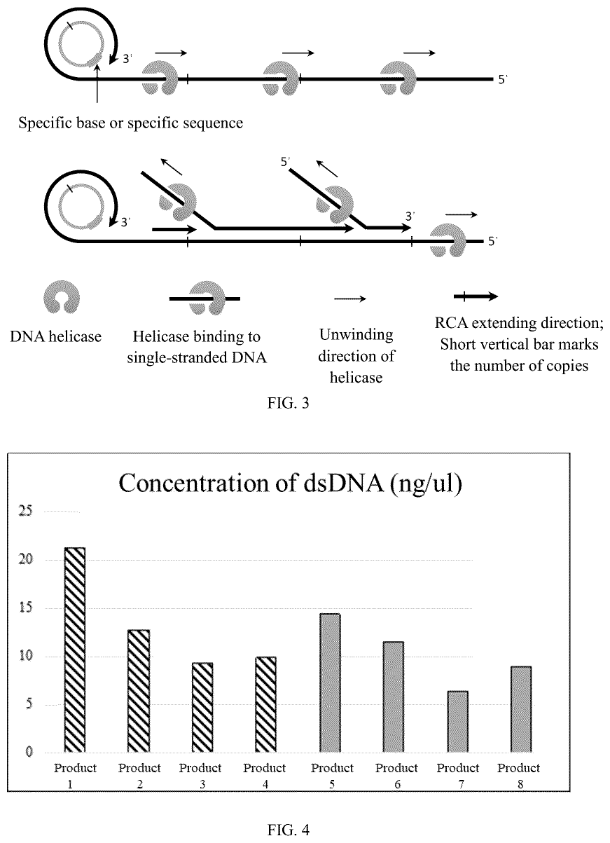

In a preferable example of the present disclosure, a helicase may be added to participate in the RCA, to obtain more “fluffy” RCA products. FIG. 3 is a schematic diagram illustrating a mechanism of a helicase in the 3′-5′ direction binding to a sequence amplified by rolling circle amplification. According to examples of the present disclosure, helicases may be added during or after the RCA. Without intending to be bound to any specific theory, helicase uses the energy released by ATP hydrolysis to change its conformation and breaks hydrogen bonds between the double strands of DNA. Helicase binds specifically to a single-stranded DNA and begins to unwind along the 3′ to 5′ direction under the action of ATP. The addition of helicases during or after RCA reactions helps maintain RCA products in relatively simple single-stranded form, creating conditions for subsequent reverse RCA reactions.

Helicase possesses directionality, and a single-stranded DNA helicase along the 3′ to 5′direction (i.e. Type A alpha Helicase family) is adopted as an example to illustrate the principle of the present disclosure, as shown in FIG. 3. In specific examples of the present disclosure, Tte UvrD helicase (NEB) may be used. Similarly, any type A helicase unwinding in the 3′ to 5′ direction, e.g. Rep, Heli308, PcrA, RecD2, etc., may all be used as alternatives to examples of the present disclosure and the present disclosure is intended to include these helicases.

In the step 3, the single-stranded circular DNA is opened.

In examples of the present disclosure, the single-stranded circular DNA is disconnected at the specific base or specific sequence through the biochemical reaction, to form a single-stranded linear DNA. For instance, after a period of RCA reaction, appropriate enzymes may be introduced to cut the special bases or specific sequences in the circular DNA molecule, thus to open the single-stranded circular DNA. The ring-opening mode is related to the special base or specific sequence in the circular DNA molecule, for example, according to the latter selecting the appropriate enzyme, and the ring-opening schematic diagram is shown in FIG. 2. It would be understood that after the single-stranded circular DNA is opened, the polymerase involved in the RCA reaction (such as Phi29) will fall off the template when reaches the gap position due to lack of template, thus terminating the RCA reaction.

In the step 4, the amplification in the opposite direction of RCA is performed.

In this step, the single-stranded linear DNA, as a second primer, is subjected to a reverse RCA replication, to obtain an amplified double-stranded sequence. Specifically, when the single-stranded circular DNA is opened, the single-stranded linear DNA, generated from the single-stranded circular DNA, shows a naked 3′ end which may be recognized by polymerase. At this time, by supplementing RCA reaction buffer and required polymerase, the single-stranded linear DNA may be taken as a primer, and begins to perform another RCA reaction in the opposite direction of the initial RCA, i.e., reverse RCA reaction, as shown in FIG. 2. It would be understood that this reverse RCA reaction does not require any additional primers or annealing, where the original template could be used as the primer for secondary strand synthesis, obtaining a complete RCA double-strand synthesis product in the same reaction system.

### Example 1: DNA Fragment as Template to Verify the Feasibility of the Method Provided by the Present Disclosure

### 1.1 Preparation of DNA Template

1.1.1 In order to prepare template DNA-1, a PCR-1 system was prepared as follow: 5 μL of 10× Standard Taq Reaction Buffer (NEB), 1 μL of 10 mM dNTPs (NEB), 0.25 μL of Taq DNA Polymerase (NEB), 0.25 μM of GAPDH500Fp-1 primer (Beijing Liuhe), 0.25 μM of GAPDH500R primer (Beijing Liuhe), and 0.01 ng of human transcriptome cDNA were added to obtain 50 μL reaction system. The obtained PCR mixture was placed in a PCR amplifier to perform the following procedure: 98° C. for 2 minutes; 95° C. for 30 seconds, 56° C. for 30 seconds and 72° C. for 2 minutes, for 20 cycles; finally 72° C. for 10 minutes for incubation, and 4° C. for hold. After that, the PCR product was purified with 0.8× AMPure magnetic beads (Beckman) to obtain the template DNA-1, where purification protocol referred to instructions of AMPure magnetic beads.

The GAPDH500Fp-1 primer has the following sequence: 5′-Phosphate-AGCCACAUCGCUCAGACAC-3′ (SEQ ID NO: 1); and
the GAPDH500R primer has the following sequence: 5′-GAGGCATTGCTGATGATCTTG-3′ (SEQ ID NO: 2).

(SEQ ID NO: 2)
5′-GAGGCATTGCTGATGATCTTG-3′.

In order to prepare template DNA-2, a PCR-2 system was prepared as follow: 5 μL of 10× Standard Taq Reaction Buffer (NEB), 1 μL of 10 mM dNTPs (NEB), 0.25 μL of Taq DNA Polymerase (NEB), 0.25 μM of GAPDH500Fp-2 primer (Beijing Liuhe), 0.25 μM of GAPDH500R primer (Beijing Liuhe), and 0.01 ng of human transcriptome cDNA were added to obtain 50 μL reaction system. The obtained PCR mixture was placed in a PCR amplifier to perform the following procedure: 98° C. for 2 minutes; 95° C. for 30 seconds, 56° C. for 30 seconds and 72° C. for 2 minutes, for 20 cycles; finally 72° C. for 10 minutes for incubation, and 4° C. for hold. After that, the PCR product was purified with 0.8× AMPure magnetic beads (Beckman) to obtain the template DNA-2, where purification protocol referred to instructions of AMPure magnetic beads.

The GAPDH500Fp-2 primer has the following sequence: 5′-Phosphate-AGCCACAICGCICAGACAC-3′ (SEQ ID NO: 3); and
the GAPDH500R primer has the following sequence:

(SEQ ID NO: 4)
5′-GAGGCATTGCTGATGATCTTG-3′.

1.1.2 In this example, the GAPDH500Fp-1 primer used was modified with specific bases, dU, which were introduced into an adapter through PCR reaction. The GAPDH500Fp-2 primer used in this example was modified with specific bases, I, which were introduced into an adapter through PCR reaction. By this means, the template DNA may also be modified with, including but not limited to an AP site, a methylation site, a specific sequence and the like.

1.1.3 With purification, the template DNA-1 obtained has the following sequence (SEQ ID NO: 5):

5′-AGCCACAUCGCUCAGACACCATGGGGAAGGTGAAGGTCGGAGTC
AACGGATTTGGTCGTATTGGGCGCCTGGTCACCAGGGCTGCTTTTAA
CTCTGGTAAAGTGGATATTGTTGCCATCAATGACCCCTTCATTGACC
TCAACTACATGGTTTACATGTTCCAATATGATTCCACCCATGGCAAA
TTCCATGGCACCGTCAAGGCTGAGAACGGGAAGCTTGTCATCAATGG
AAATCCCATCACCATCTTCCAGGAGCGAGATCCCTCCAAAATCAAGT
GGGGCGATGCTGGCGCTGAGTACGTCGTGGAGTCCACTGGCGTCTTC
ACCACCATGGAGAAGGCTGGGGCTCATTTGCAGGGGGGAGCCAAAAG
GGTCATCATCTCTGCCCCCTCTGCTGATGCCCCCATGTTCGTCATGG
GTGTGAACCATGAGAAGTATGACAACAGCCTCAAGATCATCAGCAAT
GCCTC-3′.

With purification, the template DNA-2 obtained has the following sequence (SEQ ID NO: 6):

5′-AGCCACAICGCICAGACACCATGGGGAAGGTGAAGGTCGGAGTC
AACGGATTTGGTCGTATTGGGCGCCTGGTCACCAGGGCTGCTTTTAA
CTCTGGTAAAGTGGATATTGTTGCCATCAATGACCCCTTCATTGACC
TCAACTACATGGTTTACATGTTCCAATATGATTCCACCCATGGCAAA
TTCCATGGCACCGTCAAGGCTGAGAACGGGAAGCTTGTCATCAATGG
AAATCCCATCACCATCTTCCAGGAGCGAGATCCCTCCAAAATCAAGT
GGGGCGATGCTGGCGCTGAGTACGTCGTGGAGTCCACTGGCGTCTTC
ACCACCATGGAGAAGGCTGGGGCTCATTTGCAGGGGGGAGCCAAAAG
GGTCATCATCTCTGCCCCCTCTGCTGATGCCCCCATGTTCGTCATGG
GTGTGAACCATGAGAAGTATGACAACAGCCTCAAGATCATCAGCAAT
GCCTC-3′.

1.1.4 The template DNA-1 and the template DNA-2 each, obtained in step 1.1.1 was subjected to cyclization, in which the reaction system was prepared as follows: 12.5 μL of 0.1 M TE buffer, 2.5 μL of GAPDH500splint primer (20 μM) and 330 ng of the template DNA-1 or DNA-2 above were added to obtain 48 μL reaction system. After mixing well, the reaction system was placed in a PCR amplifier, for incubating at 95° C. for 3 minutes, and then immediately transferred to ice, continuing to incubate for another 10 minutes. After that, 6 μL of 10× T4 DNA ligation buffer (NEB, M0202S), 0.6 μL of 100 mM ATP (NEB, P0756S), and 0.2 μL of T4 DNA ligase (600 U/μL, NEB, M0202S) were added into the 48 μL reaction system above, then making up to a total volume of 60 μL with water. The reaction system was then placed in the PCR amplifier and incubated at 37° C. for 1 hour.

The GAPDH500splint primer has the following sequence:

(SEQ ID NO: 7)
5′-AGCGATGTGGCTGAGGCATTGCTG-3′.

1.1.5 4 μL of respective reaction products of template DNA-1 and DNA-2 obtained by step 1.1.4 were transferred into new PCR tubes for later use, and the respective remaining 56 μL reaction products were added with 0.4 μL of 10× T4 DNA ligation buffer (NEB, M0202S), 1.95 μL of Exonuclease I (20 U/μL, NEB, M0293S) and 0.65 μL of Exonuclease III (100 U/μL, NEB, M0206S), then making up to a total volume of 60 μL with water. The resulting reaction solution was placed in a PCR amplifier and incubated at 37° C. for 30 minutes.

1.1.6 The cyclization products obtained by step 1.1.5 were purified with 2.5× AMPure magnetic beads (Beckman) and then quantified with Qubit ssDNA Kit.

1.2 RCA Involved with Helicase (Heli-RCA)

1.2.1 Helicase is a DNA binding protein that requires ATP (adenosine triphosphate) for energy supply, and different helicases have different directionality. In this example, Tte UvrD helicase (NEB), an ATP-dependent and possessing 3′ to 5′ directionality, was used. Tte UvrD helicase binds spontaneously to a single-stranded DNA and present no unwinding activity in the absence of ATP. With the addition of ATP during RCA, in this example, the unwinding and RCA proceeded simultaneously, where the helicase unwound the RCA product along its 3′ to 5′ direction under the action of ATP. The introduction of helicases could minimize the secondary structures of the RCA products.

1.2.2 A RCA reaction solution was prepared as follows: 10 μL of RCA buffer, 20 μL of RCA enzyme mix1 and 2 μL of RCA enzyme mix2 (MGIEasy stLFR Library Preparation Kit) were added to a PCR tube, followed by 4 ng of DNA-1 and DNA-2 cyclization products obtained from step 1.1.6, respectively, then making up to 37.5 μL with water. The resulting reaction solution was placed in a PCR amplifier to incubate at 30° C. for 5 minutes, then immediately placed in ice, and added with 0.5 μL of Tte UvrD helicase (13.4 μM) and 2 μL of ATP (0.1 M, NEB) were added respectively. After sufficient mixing, the reaction solutions were placed in a PCR amplifier and incubated at 30° C. for 25 minutes, then heated to 65° C., and incubated for 15 minutes.

1.2.3 Control groups were set with similar reaction conditions and reaction systems, differing in that the Tte UvrD helicase was replaced by equal amount of molecular water in the control groups.

### 1.3 Ring Opening

1.3.1 20 μL of RCA products of DNA-1 obtained by step 1.2 were transferred into a new PCR tube, and added with the following reagents: 3 μL of NEB buffer 2, 2 μL of UDG (5 U/μL, NEB) and 3 μL of APE1 (10 U/μL, NEB), then making up to 30 μL with water. The resulting reaction solution was placed in a PCR amplifier and incubated at 37° C. for 30 minutes. Similarly, 20 μL of RCA products of the control group of DNA-1 obtained by step 1.2 were transferred into a new PCR tube, and also added with the following reagents: 3 μL of NEB buffer 2, 2 μL of UDG (5 U/μL, NEB) and 3 μL of APE1 (10 U/μL, NEB), then making up to 30 μL with water. The resulting reaction solution of the control group was placed in a PCR amplifier and incubated at 37° C. for 30 minutes.

1.3.2 A control test 1 versus the reaction in step 1.3.1 was set, where only 3 μL of NEB buffer 2 were added, followed by making up the system to 30 μL with water. The control system 1 was placed in a PCR amplifier and incubated at 37° C. for 30 minutes.

1.3.3 20 μL of RCA products of DNA-2 obtained by step 1.2 were transferred into a new PCR tube, and added with the following reagents: 3 μL of NEB buffer 4 and 1 μL of Endonuclease V (10 U/μL, NEB), then making up to 30 μL with water. The resulting reaction solution was placed in a PCR amplifier and incubated at 37° C. for 30 minutes. Similarly, 20 μL of RCA products of the control group of DNA-2 obtained by step 1.2 were transferred into a new PCR tube, and also added with the following reagents: 3 μL of NEB buffer 4 and 1 μL of Endonuclease V (10 U/μL, NEB), then making up to 30 μL with water. The resulting reaction solution of the control group was placed in a PCR amplifier and incubated at 37° C. for 30 minutes.

1.3.4 A control test 2 versus the reaction in step 1.3.3 was set, where only 3 μL of NEB buffer 4 were added, followed by making up the system to 30 μL with water. The control system 2 was placed in a PCR amplifier and incubated at 37° C. for 30 minutes.

1.3.5 The reaction solution of DNA-1 was digested by UDG/APE1, as base “dU” was introduced into the template DNA-1 during template preparation; while the reaction solution of DNA-2 was digested by Endonuclease V, because base “I” was introduced into the template DNA-2 during template preparation. For different bases or specific sequences, different ring opening schemes may be selected as required, which will not be described in detail in this example.

1.4 Reverse RCA Reaction, i.e. Reverse Rolling Circle Replication (RRCR)

The single-stranded linear DNA, as a primer, was subjected to the reverse RCA replication, to obtain an amplified double-stranded sequence. Specifically, the reaction product solutions of DNA-1 and DNA-2 after ring opening, and reaction product solutions of their control tests respective, obtained by step 1.3, were added with the following reagents individually: 20 μL of RCA enzyme mix1 and 2 μL of RCA enzyme mix2 (MGIEasy stLFR library preparation kit). After sufficient mixing, the reaction solutions were placed in a PCR amplifier and incubated at 30° C. for 30 minutes, then heated to 65° C. for incubating for 15 minutes, and held at 4° C.

### Experimental Results

The processes above may be represented as the following steps 1 to 5 briefly, yielding a total of 8 products, which were labeled as products 1 to 8, respectively.

| Step 1 | PCR amplification with a primer containing dU | | | | PCR amplification with a primer containing I | | | |
|---|---|---|---|---|---|---|---|---|
| Step 2 | Cyclization to obtain a circular template DNA-1 | | | | Cyclization to obtain a circular template DNA-2 | | | |
| Step 3 | Heli-RCA | RCA | Heli-RCA | RCA | Heli-RCA | RCA | Heli-RCA | RCA |
| Step 4 | Ring | Ring | Without | Without | Ring | Ring | Without | Without |
| | opening | opening | ring opening | ring opening | opening | opening | ring opening | ring opening |
| Step 5 | RRCR | RRCR | RRCR | RRCR | RRCR | RRCR | RRCR | RRCR |
| Product | Product 1 | Product 2 | Product 3 | Product 4 | Product 5 | Product 6 | Product 7 | Product 8 |

The results are shown in FIG. 4, in which the products 1 to 8 had concentrations (ng/μL) of dsDNA for 21.30, 12.76, 9.33, 9.87, 14.38, 11.55, 6.42 and 8.96, respectively. As the RCA was involved with helicase, the secondary structures of the products were simpler than that of conventional RCA products, and thus the secondary strand synthesis were more complete during the reverse RCA replication, thereby yielding more dsDNA products with the RCA reverse replication. Therefore, the above results show that the introduction of helicase can significantly improve the efficiency of secondary strand synthesis.

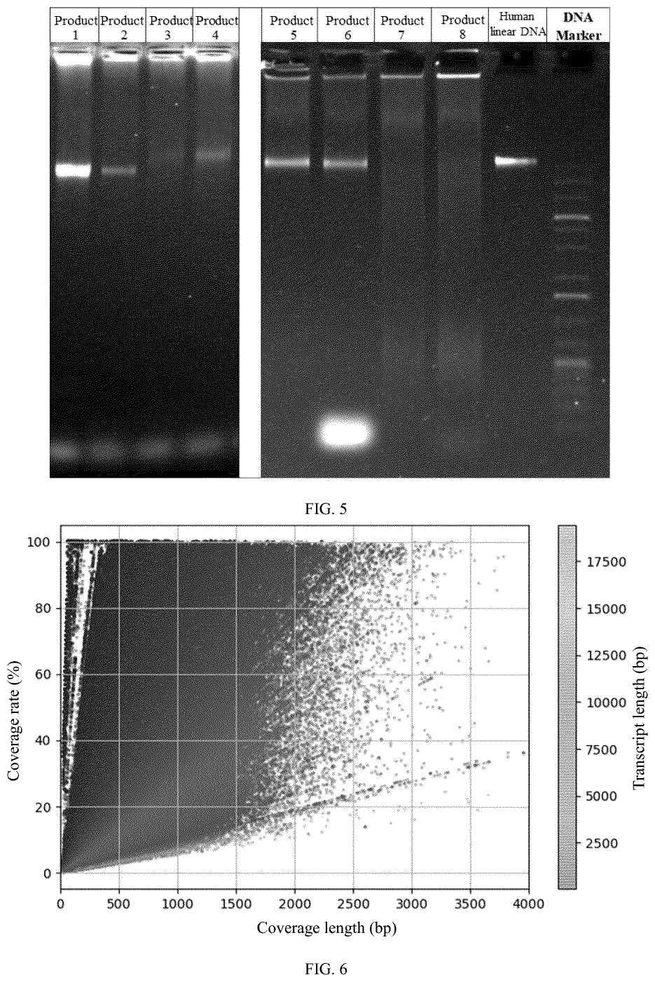

Conventional RCA products and products after secondary strand synthesis based on the RCA present complex secondary structures, and will be stuck in the gel wells, with a small number of them leaving the gel wells to form smears. With regard to the linear double-stranded RCA product, of which structure is similar to that of ordinary DNA molecule having double strands, it can leave gel wells during agarose gel electrophoresis. Accordingly, secondary strand synthesis of the products may be visually observed with agarose gel electrophoresis, which is shown in FIG. 5.

### Example 2: Construction and Sequencing of mRNA Full-Length Transcript Library Based on MGIEasy stLFR Library Preparation Kit

2.1 Preparation and Enrichment of mRNA Full-Length Transcript (cDNA) In view of the stLFR (single tube Long Fragment Read, provided by MGI) technology sequencing reads from 10 k to 300 k, and the average length of human cDNA being about 2 kb, this example gathered a plurality of copies of the full-length cDNA sequence into one sequence with the RCA method provided in the present disclosure, so as to realize the preparation and enrichment of full-length cDNA.

2.1.1 A capture sequence for capturing mRNA, TSO primer for reverse transcription, ISO primer, oligo dT sequence for rolling circle amplification, and TnSplint primer for circularization were synthesized and each of them was dissolved to a concentration of 100 μM with TE solution and stored at −20° C. for later use. In this example, the following steps were performed using an input amount of 1 μg of total RNA as an example.

The capture sequence has the following sequence: 5′-AAGCdUdUCGTAGCCATGTCGTTCTGCGNNNNNNNNNNTTTTTTTTTTTTTTTTTTTT TV-3′ (SEQ ID NO: 8), in which N refers to A/T/C/G, and V refers to A/G/C.

The TSO primer has the following sequence: 5′-AAGCdUdUCGTAGCCATGTCGTTCTGrGrGrG-3′ (SEQ ID NO: 9), in which rG refers to a RNA base G, i.e., guanine ribonucleotide.

The ISO primer has the following sequence: 5′-AAGCdUdUCGTAGICATGTIGTTCTG-3′ (SEQ ID NO: 10).

The oligo dT sequence has the following sequence: 5′-TTTTTTTTTTTTTTTT-3′ (SEQ ID NO: 11).

2.1.2 To 1 μL of human total RNA (1 μg) was added 5 μL of dNTP (10 mM) and 1 μL of the capture sequence (50 μM), and placed in a PCR amplifier at 72° C. for 3 minutes, and then removed to ice immediately for 1 minute. After that, a reverse transcriptase reaction mixture containing 1 μL of reverse transcriptase (SuperScript II reverse transcriptase, 200 U/μL, Invitrogen), 0.5 μL of RNaseOUT™ (RNase inhibitor, 40 U/μL, Invitrogen), 4 μL of 5× Superscript II first-strand buffer (5-fold reverse transcriptase II buffer; 250 mM Tris-HCl, pH 8.3; 375 mM KCl; 15 mM MgCl₂, Invitrogen), 0.5 μL of DTT (100 mM, Invitrogen), 6 μL of MgCl₂ (25 mM, Invitrogen) and 0.5 μL of TSO primer (100 μM) were added, with water to make up to 20 μL. The obtained reverse transcription reaction system was placed in a PCR amplifier for reverse transcription reaction with the following procedures: (i) 42° C. for 90 minutes; (ii) 50° C. for 2 minutes; (iii) 42° C. for 2 minutes; wherein (ii) to (iii) were run for 10 cycles.

2.1.3 Subsequent to the reverse transcription reaction above, 50 μL of 2× KAPA HiFi HotStart Ready Mix containing 5 mM MgCl₂, 0.6 mM of each dNTP and 1 U KAPA HiFi HotStart DNA Polymerase, as well as 5 μL of ISO primer (10 μM) were added and the volume was made up to 100 μL with water. The obtained amplification reaction system was subjected to the following condition for amplification: (i) 98° C. for 3 minutes; (ii) 98° C. for 20 seconds; (iii) 67° C. for 15 seconds; (iv) 72° C. for 6 minutes; and (v) 72° C. for 5 minutes; wherein the steps (ii) to (v) were repeated for 10-20 cycles.

2.1.4 Subsequent to the amplification reaction of step 2.1.3 above, the amplified product was purified with 200 μL of XP magnetic beads (Agencourt AMPure XP-Medium, A63882, AGENCOURT), and the purification method is described in the standard operating procedures provided by the manufacturer.

2.1.5 After purification in step 2.1.4, to the purified product was added 1 μL of USER enzyme (1 U/μL, NEB) and 3 μL of 10× stTaq Buffer (10-fold standard Taq buffer, 100 mM Tris-HCl, 500 mM KCl, 15 mM MgCl₂), and the volume was made up to 30 μL with water. The resulting reaction system was placed in a PCR amplifier for reaction at 37° C. for 1 hour, during which the USER enzyme cut cDNA to present sticky ends to facilitate subsequent ligation cyclization.

2.1.6 After the reaction in step 2.1.5, 5 μL of 10× TA Buffer were added to the reaction system, making up to 50 μL with water. The reaction system was placed in a PCR amplifier for reaction at 70° C. for 30 minutes, followed by water bath at room temperature for 20 minutes.

2.1.7 After the reaction in step 2.1.6, 2 μL of 10× TA Buffer, 0.752 μL of 0.1 M ATP and 0.1 μL of T4 DNA Ligase (Enzymatics, 600 U/μL) were added to the reaction system, making up to 55 μL with water and then incubating at room temperature for 2 hours.

2.1.8 After the reaction in step 2.1.7, the reaction product was purified with 55 μL of XP magnetic beads (Agencourt AMPure XP-Medium, A63882, AGENCOURT), and the purification method is described in the standard operating procedures provided by the manufacturer.

2.1.9 Subsequent to the purification in step 2.1.8, to the purified product was added 3 μL of 10× TA Buffer, 1.95 μL of Exonuclease I (20 U/μL, NEB, M0293S) and 0.65 μL of Exonuclease III (100 U/μL, NEB, M0206S), making up to 30 μL with water and then reacting at 37° C. for 30 minutes in a PCR amplifier.

2.1.10 After the reaction in step 2.1.9, the reaction product was purified with 60 μL of XP magnetic beads (Agencourt AMPure XP-Medium, A63882, AGENCOURT), and the purification method is described in the standard operating procedures provided by the manufacturer.

So far, circularization of the single-stranded full-length transcript was completed.

2.1.11 A rolling circle amplification reaction mixture was prepared with 4 μL of oligo dT (50 μM) and 40 μL of 10× phi29 buffer (10-fold concentration of phi29 buffer), making up to 200 μL with water.

2.1.12 To the purified product obtained by step 2.1.10 was added 20 μL of rolling circle amplification reaction mixture prepared in step 2.1.11, making up to 40 μL with water. The reaction mixture was then subjected to the following procedures: 95° C. for 1 minute; 65° C. for 1 minute; and 40° C. for 1 minute. After that, the product was placed in ice, during this the oligo dT, with annealing, bond to the purified product which was served as template.

2.1.13 To the product of step 2.1.12 was added 40 μL of Make DNB Buffer (MGI, P093) and 4 μL of RCA Enzyme Mix (MGI, P094) and then placed in a PCR amplifier at 30° C. for 2 minutes, and then removed to ice immediately, and 1 μL of Tte UvrD helicase (NEB, M1202S) and 1 μL of ATP (10 mM) were added, reacting in a PCR amplifier at 30° C. for 30 minutes and then 65° C. for 10 minutes.

2.1.14 After the reaction of step 2.1.13, concentration was measured using a single-strand concentration test kit (Lifetech).

2.1.15 To 100 ng of the product obtained in step 2.1.13 were added 2 μL of 10×NEB buffer 4 (10-fold concentration of NEB buffer 4), 2 μL of NEB Endonuclease V, and water to make up to 20 μL, and then placed in a PCR amplifier to perform the following procedure: 37° C. for 30 minutes and 65° C. for 10 minutes. After that, 20 μL of Make DNB Buffer (BGI) and 2 μL of RCA Enzyme Mix (BGI) were added and the reaction system was place in a PCR amplifier, reacting at 30° C. for 30 minutes and 65° C. for 10 minutes.

2.1.16 After the reaction of step 2.1.15, the obtained product was purified with 50 μL of XP magnetic beads (Agencourt AMPure XP-Medium, A63882, AGENCOURT), and the purification method is described in the standard operating procedures provided by the manufacturer. Until this, preparation and enrichment of mRNA full-length transcript (double-stranded cDNA) were completed.

### 2.2 Preparation and Sequencing of Short Fragments Having Molecular Tags

2.2.1 The mRNA full-length transcript (double-stranded cDNA) obtained in step 2.1 was subjected to preparation of LFR library with MGIEasy stLFR library preparation kit, and the library construction process was carried out according to the instructions of the MGIEasy stLFR kit.

2.2.2 The prepared library in step 2.2.1 was subjected to single-stranded cyclization so as to be sequenced on BGISEQ-500, details for which referred to the cyclization of BGISEQ-500 standard DNA fragment library preparation process. The short fragment information obtained by sequencing was restored to long cDNA information through molecular tags, thereby obtaining mRNA expression level.

### 2.3 Experimental Results

2.3.1 The sequencing results are shown in the following Table 1.

TABLE 1
Summary of sequencing reads

| | Number of raw | Number of clean | Number of reads | Number of reads |
|---|---|---|---|---|
| Sample | reads | reads | with low quality | with too many N |
| Sample 1 | 135551886 | 132782478 | 22704 | 60 |

| | Total number | Total number | Number of | Number of | Number of |
|---|---|---|---|---|---|
| | of mapping | of non- | unique | multi-mapping | accurate |
| Sample | reads | mapping reads | mapping reads | reads | mapping reads |
| Sample 1 | 110604665 | 22539943 | 110604665 | 0 | 89034707 |
| | (83.07%) | (16.93%) | (83.07%) | (0.00%) | (66.87%) |

2.3.2 Assembly results of sequencing reads

FIG. 6 shows a relationship between coverage lengths versus coverage rate in the assembly of Sample 1, in the form of a point diagram, where the x-axis indicates coverage length of the assembled contigs to the transcript, the y-axis indicates the coverage percentage of the contigs to the transcript, and the color from black to gray indicates transcript length from short to long. The results in FIG. 6 show that the assembly length of each transcript reached 100%, and the longest transcript coverage length reached about 4000 bp.

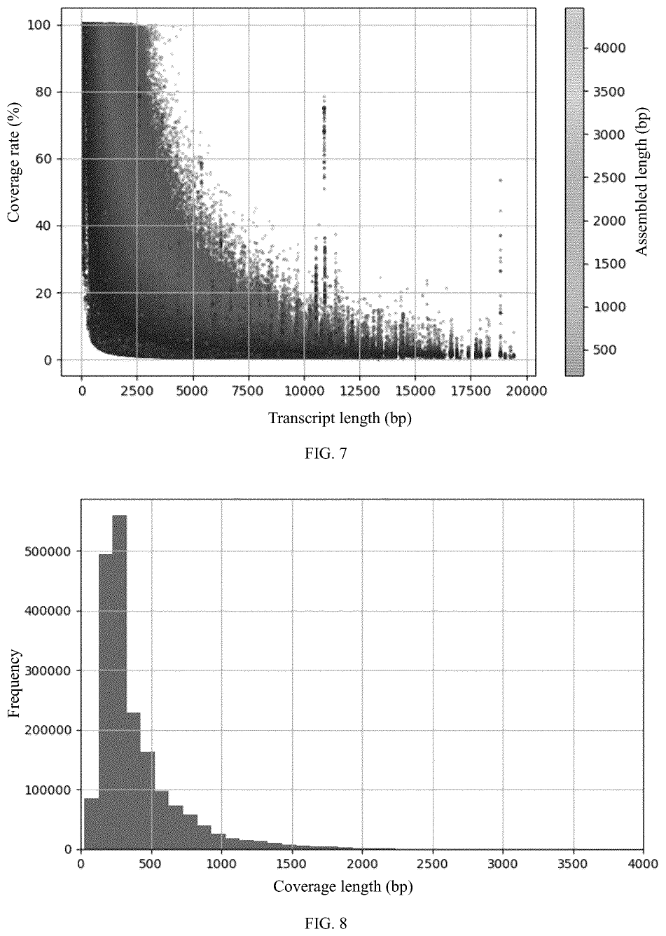

FIG. 7 shows a relationship between transcript lengths versus coverage rate of assembly of Sample 1, in the form of a point diagram, where the x-axis indicates the length of the transcript, the y-axis indicates coverage rate of contigs to the transcript, and the color from black to gray indicates assembly length from short to long. The results in FIG. 7 show that the full length of most transcripts could be obtained by assembly.

FIG. 8 shows a distribution of the coverage lengths in assembly of Sample 1, in the form of a histogram to present the coverage lengths of the assembled contigs to the transcript, in which the x-axis indicates the coverage length of the assembled contigs to the transcript, and the y-axis indicates frequency. The results in FIG. 8 show that it is of high frequency to the contigs to be assembled into a full length transcript.

1. A method for obtaining a double-stranded sequence by single-stranded rolling circle amplification, comprising the following steps:
i) subjecting a single-stranded circular DNA to the rolling circle amplification with a first primer to obtain an amplified sequence, wherein the first primer is complementary to a partial region of the single-stranded circular DNA, and the single-stranded circular DNA possesses a disconnection mechanism to open the single-stranded circular DNA;
ii) opening the single-stranded circular DNA through the disconnection mechanism, to obtain a single-stranded linear DNA; and
iii) performing amplification with the single-stranded linear DNA as a second primer and the amplified sequence obtained in step i) as a template, to obtain an amplified double-stranded sequence.

2. The method according to claim 1, wherein the single-stranded circular DNA is obtained by cyclizing a DNA sample or a cDNA sample and introducing a specific base or a specific sequence into the cyclized single-stranded circular DNA by PCR or adapter connection.

3. The method according to claim 1, wherein the first primer is a DNA primer or an RNA primer.

4. The method according to claim 1, wherein the disconnection mechanism is to open the single-stranded circular DNA through a specific region in the single-stranded circular DNA, wherein the specific region is broken in response to a biochemical reaction.

5. The method according to claim 4, wherein the specific region comprises one or both of the specific base and specific sequence.

6. The method according to claim 5, wherein the specific base is a hypoxanthine, a dU, an RNA base, an AP site, or a methylation site.

7. The method according to claim 6, wherein the specific base is the hypoxanthine, which is digested and cleaved by endonuclease V to open the single-stranded circular DNA.

8. The method according to claim 6, wherein the specific base is the dU, which is recognized and cleaved by uracil-DNA glycosylase (UDG) or apyrimidinic endonuclease 1 (APE1), to open the single-stranded circular DNA.

9. The method according to claim 6, wherein the specific base is the RNA base, which is recognized and cleaved by RNaseA or RNaseH, to open the single-stranded circular DNA.

10. The method according to claim 6, wherein the specific base is the AP site, which is recognized and cleaved by APE1, to open the single-stranded circular DNA.

11. The method according to claim 6, wherein the specific base is the methylation site of a methylated cytosine (C), which is treated by APOBEC deaminase, ten-eleven transmethylase 2 (TET2), or sodium bisulfite to convert the methylated C to a dU, which is recognized and cleaved by UDG or APE1, to open the single-stranded circular DNA.

12. The method according to claim 5, wherein the specific sequence is a restriction endonuclease recognition site or a protein-specific binding site.

13. The method according to claim 12, wherein the restriction endonuclease recognition site is a region rich in AT sequences.

14. The method according to claim 12, wherein the protein-specific binding site is a guide RNA recognition region of a CRISPR/Cas gene editing system.

15. The method according to claim 1, comprising: adding a single-stranded DNA binding protein, a pyrophosphatase, and TE buffer during or after subjecting the single-stranded circular DNA to the rolling circle amplification.

16. The method according to claim 1, comprising: adding a helicase during or after subjecting the single-stranded circular DNA to the rolling circle amplification.

17. The method according to claim 16, wherein the helicase is a type A helicase unwinding in a 3′ to 5′ direction.

18. A method for constructing a nucleic-acid sequencing library, comprising:
i) obtaining an amplified double-stranded sequence according to a method of claim 1; and
ii) subjecting the amplified double-stranded sequence to sequencing library construction, to obtain the nucleic-acid sequencing library.

19. (canceled)

20. The method according to claim 18, wherein the nucleic-acid sequencing library is an mRNA full-length transcript library.

21. A sequencing method, comprising:
i) obtaining a nucleic-acid sequencing library by a method according to claim 18; and
ii) sequencing the nucleic-acid sequencing library.

22. (canceled)
